(19) United States
(12) Patent Application Publication
(10) Pub. No.: US 2006/0194724 A1
(43) Pub. Date: Aug. 31, 2006

(54) Methods and systems for nerve regeneration

(76) Inventors: Todd K. Whitehurst, Santa Clarita, CA (US); James P. McGivern, Stevenson Ranch, CA (US); Rafael Carbunaru, Studio City, CA (US); Kelly H. McClure, Simi Valley, CA (US); Kristen N. Jaax, Saugus, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/066,993

(22) Filed: Feb. 25, 2005

(51) Int. Cl. A61K 38/18; A61N 1/18

(52) U.S. Cl. 514/12; 607/42

(57) ABSTRACT

An exemplary method of regenerating a nerve within a patient includes implanting a system control unit within the patient and applying a stimulus to the nerve with the system control unit in accordance with one or more control parameters. The stimulus is configured to promote regeneration of the nerve. An exemplary system for regenerating a nerve within a patient includes a system control unit configured to apply a stimulus to the nerve in accordance with one or more control parameters. The system control unit is implanted within the patient and the stimulus promotes the regeneration of the nerve.

### BACKGROUND

Recent estimates indicate that hundreds of thousands of Americans suffer peripheral nerve injuries every year. These injuries vary in severity and include, but are not limited to, inflammation, compression, transection, ischemia, degeneration, and radiation-induced damage. Peripheral nerve injuries may result in discomfort, pain, or dysfunction in corresponding parts of the body.

For example, many males who undergo prostate surgery (e.g., radical retropubic prostatectomy (RRP)) suffer injuries to the cavernous and/or pudendal nerves during the course of the operation. The cavernous and pudendal nerves are essential in achieving and maintaining a penile erection. Thus, erectile dysfunction is a common complication for thousands of males who undergo prostate surgery.

Other examples of common peripheral nerve injuries include traumatic injuries to the brachial plexus caused by falls and automobile and motorcycle accidents, nerve compression injuries caused by tumors or other masses, and nerve transection injuries caused by knife wounds. Peripheral nerve injuries may be caused by a number of additional and/or different factors.

### SUMMARY

An exemplary method of regenerating a nerve within a patient includes implanting a system control unit within the patient and applying a stimulus to the nerve with the system control unit in accordance with one or more control parameters. The stimulus is configured to promote regeneration of the nerve.

An exemplary system for regenerating a nerve within a patient includes a system control unit configured to apply a stimulus to the nerve in accordance with one or more control parameters. The system control unit is implanted within the patient and the stimulus promotes the regeneration of the nerve.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 is a diagram of the human nervous system according to principles described herein.

FIG. 2 illustrates an exemplary nerve injury according to principles described herein.

FIG. 3 shows that a nerve graft may be inserted in between the proximal and distal nerve stumps to facilitate or promote nerve regeneration according to principles described herein.

FIG. 4 illustrates an exemplary system control unit that may be implanted within a patient and used to apply electrical stimulation to a nerve and/or infuse one or more drugs into the nerve to promote nerve regeneration according to principles described herein.

FIG. 5 illustrates an exemplary microstimulator that may be used as the system control unit according to principles described herein.

FIG. 6 shows that one or more catheters may be coupled to the microstimulator according to principles described herein.

FIG. 7 shows a microstimulator that is coupled to a nerve graft according to principles described herein.

FIG. 8 is a flow chart illustrating an exemplary method of regenerating a damaged nerve within a patient according to principles described herein.

Throughout the drawings, identical reference numbers designate similar, but not necessarily identical, elements.

### DETAILED DESCRIPTION

Methods and systems for regenerating a damaged nerve within a patient are described herein. A system control unit (SCU) is implanted within the patient. The SCU causes a stimulus to be applied to the damaged nerve in accordance with one or more control parameters. The stimulus is configured to promote regeneration of the nerve and may include electrical stimulation of the nerve and/or stimulation via the injection of one or more drugs into the nerve.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

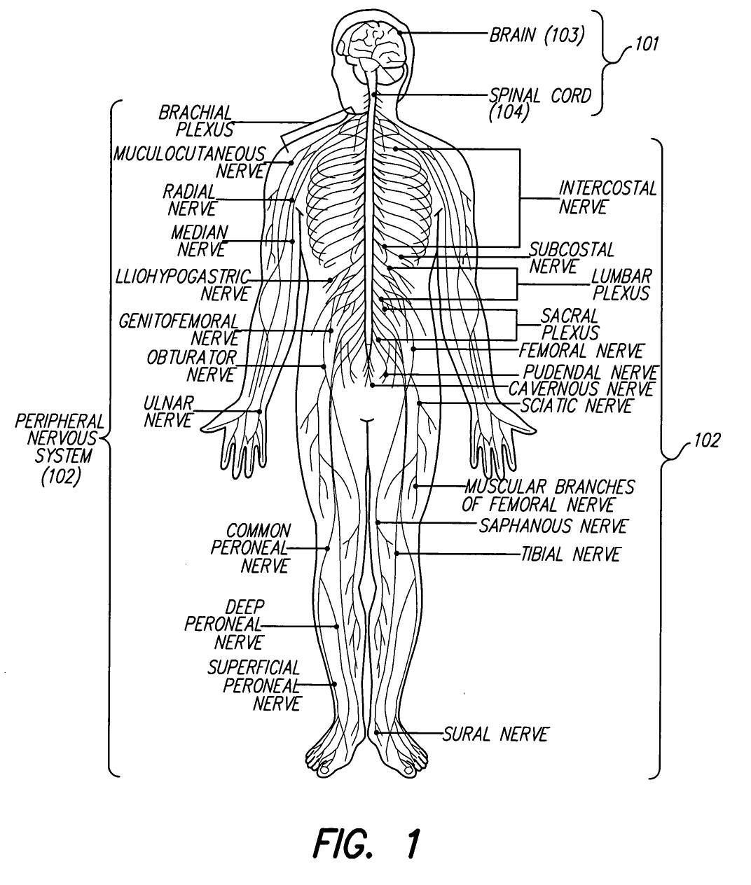

FIG. 1 is a diagram of the human nervous system. The nervous system may be divided into a central nervous system (101) and a peripheral nervous system (102). The central nervous system (101) includes the brain (103) and the spinal cord (104). The peripheral nervous system (102) includes a number of nerves that branch from various regions of the spinal cord (104). For example, the peripheral nervous system (102) includes, but is not limited to, the brachial plexus, the musculocutaneous nerve, the radial nerve, the median nerve, the lliohypogastric nerve, the genitorfemoral nerve, the obturator nerve, the ulnar nerve, the peroneal nerve, the sural nerve, the tibial nerve, the saphenous nerve, the femoral nerve, the sciatic nerve, the cavernous nerve, the pudendal nerve, the sacral plexus, the lumbar plexus, the subcostal nerve, and the intercostal nerves. Each of these peripheral nerves provides innervation to and from different parts of the body.

For example, the brachial plexus is a network of nerves that innervates the arm, forearm, and hand. The pudendal and cavernous nerves innervate the penis and clitoris and surrounding areas and are responsible for erection, orgasm, urination, and defecation. The sciatic nerve supplies motor and sensory innervation to the lower extremities.

Peripheral nerves may become damaged or injured due to a variety of causes including, but not limited to, physical impact, knife wounds, severance, surgery, or some other physical trauma. For example, many males who undergo prostate surgery suffer injuries to the cavernous and/or pudendal nerves during the course of the operation.

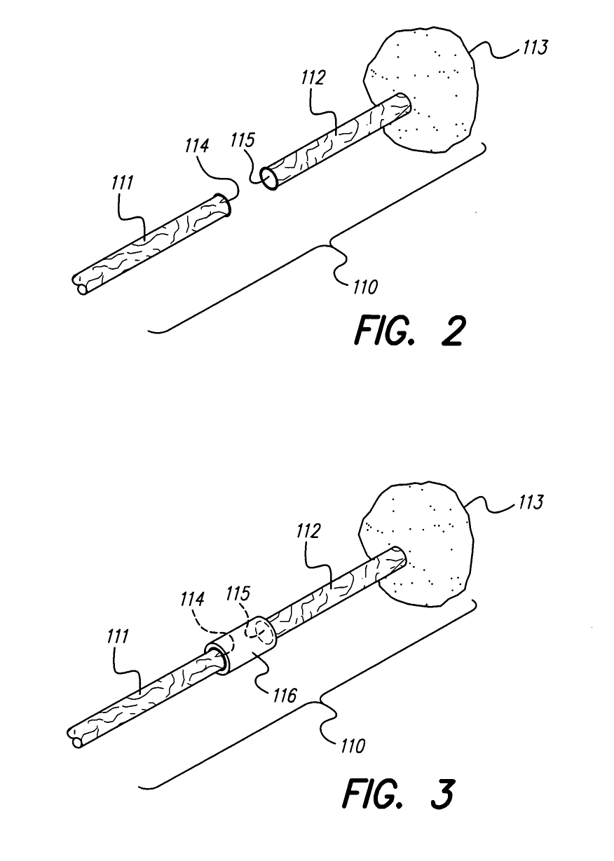

FIG. 2 illustrates an exemplary nerve injury in which a peripheral nerve (110) has been transected, or severed. In some embodiments, the transection may be caused by the surgical removal of a portion of the nerve (110). As shown in FIG. 2, the transected nerve (110) includes a proximal nerve portion (111) and a distal nerve portion (112). The proximal nerve portion (111) connects to the central nervous system (101; FIG. 1) and the distal nerve portion (112) connects to an end organ (113). The end organ (113) may be a muscle, arteriole, gland, or any other organ. As shown in FIG. 2, the transection of the nerve (110) leaves a proximal nerve stump (114) and a distal nerve stump (115).

Damaged peripheral nerves may heal in some instances through nerve regeneration. Nerve regeneration refers to renewal or physiological repair of damaged nerve tissue including, but not limited to, nerve cells, nerve axons, nerve fibers, Schwann cells, and the myelin sheath. For example, the proximal and distal nerve portions (111, 112) of the transected nerve (110) shown in FIG. 2 may regenerate and ultimately reconnect at the nerve stumps (114, 115). In some instances, the nerve stumps (114, 115) may be sutured together to facilitate this nerve generation. Alternatively, as shown in FIG. 3, a nerve graft (116) may be inserted in between the proximal and distal nerve stumps (114, 115) to facilitate or promote nerve regeneration. The nerve graft (116) may be an autologous graft from a different nerve in the patient's body. The autologous graft may be taken from the sural nerve, the genitor femoral nerve, or any other nerve within the patient's body.

Alternatively, the nerve graft (116) of FIG. 3 may be a synthetic graft or guide made out of any synthetic or naturally occurring material. For example, the nerve graft (116) may be a collagen tube impregnated with Schwann cells, stem cells, and/or any other growth factors that promote nerve regeneration. The nerve graft (116) may also contain factors to down-regulate clotting.

In some embodiments, at least one stimulus is applied to a damaged nerve to promote or facilitate faster and/or more effective nerve regeneration. The stimulus may include electrical stimulation, also known as neuromodulation. The electrical stimulation may be applied to the portion of the nerve proximal to the site where regeneration is to occur, the injured region of the nerve, and/or the portion of the nerve distal to the injury. In instances where a peripheral nerve has been transected or severed, such as the nerve (110) of FIG. 3, the electrical stimulation may be applied anywhere along the proximal nerve portion (111), the distal nerve portion (112), and/or the nerve graft (116) to promote nerve regeneration of the severed nerve (110). The electrical stimulation may also be applied directly to the proximal and distal nerve stumps (114, 115).

The stimulus applied to a damaged nerve may additionally or alternatively include drug stimulation. Therapeutic dosages of one or more drugs may be infused into a damaged nerve, into a site near the damaged nerve, or into the nerve graft or guide (116; FIG. 3) to promote faster and/or more effective nerve regeneration. These drugs may include, but are not limited to, neurotrophic factors, nerve growth factors, brain-derived neurotrophic factors (BDNF), Schwann cell products, neurotrophic tyrosine kinase type 2 (TrkB), protein kinase A (PKA), and L2/HNK-1 carbohydrate.

The electrical stimulation and/or the drug stimulation may be applied to any damaged nerve in the peripheral nervous system (102; FIG. 1). For example, the electrical stimulation and/or the drug stimulation may be applied to a damaged cavernous and/or pudendal nerve, a damaged brachial plexus, a damaged sciatic nerve, and to other damaged nerves. Although the damaged nerve is assumed to be a peripheral nerve in the examples given herein, it will be recognized that the methods and systems for applying electrical stimulation and/or drug stimulation described herein may be applied to any nerve in the central nervous system (101; FIG. 1) or the peripheral nervous system (102; FIG. 1). Furthermore, it will be recognized that the methods and systems are not limited to nerve regeneration within humans and may be applied to a damaged nerve in any animal having a nervous system.

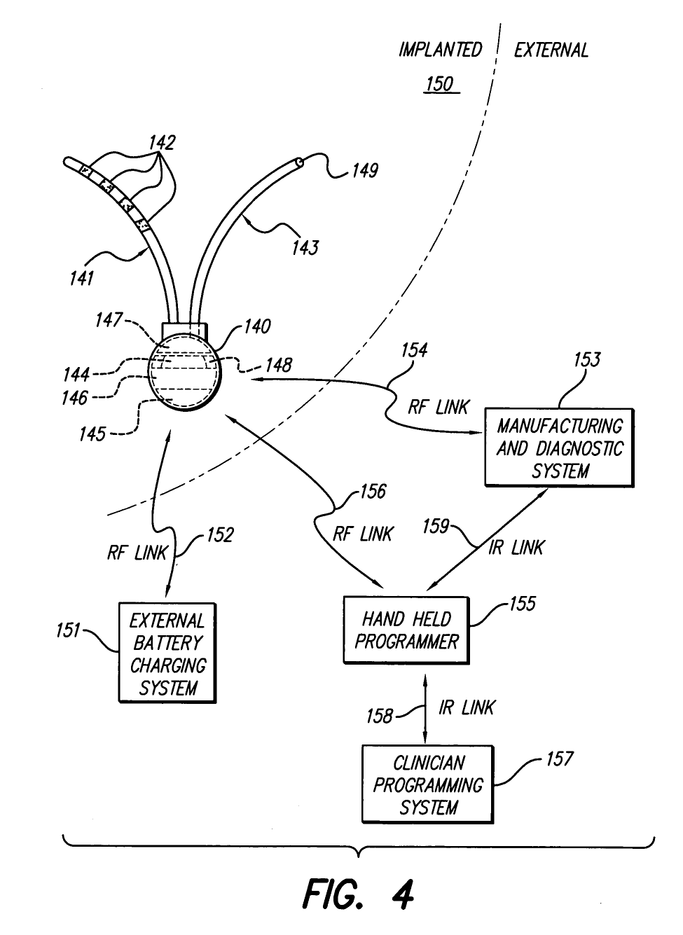

In some embodiments, the electrical stimulation and/or the drug infusion may be performed by one or more implantable system control units (SCUs). FIG. 4 illustrates an exemplary SCU (140) that may be implanted within a patient (150) and used to apply electrical stimulation to a nerve and/or infuse one or more drugs into the nerve to promote nerve regeneration. As used herein and in the appended claims, unless otherwise specifically denoted, the term “nerve” will be used to refer to any nerve tissue including, but not limited to, nerve cells, nerve axons, nerve fibers, Schwann cells, and the myelin sheath. The term “nerve” will be used herein and in the appended claims to refer to any part of the central or peripheral nervous system and will also be used herein and in the appended claims, unless otherwise specifically denoted, to refer to a nerve graft that is inserted into a transected or otherwise damaged nerve.

FIG. 4 shows that a lead (141) having a proximal end may be coupled to the SCU (140) and may include a number of electrodes (142) configured to apply electrical stimulation to a nerve. In some embodiments, the lead (141) includes anywhere between two and sixteen electrodes (142). However, the lead (141) may include any number of electrodes (142) as best serves a particular application. In some embodiments, the electrodes are alternatively inductively coupled to the SCU (140). The lead (141) may be thin (e.g., less than 3 millimeters in diameter) such that the lead (141) may be positioned near a nerve axon, for example. Alternatively, as will be described in more detail below, the SCU (140) may be leadless.

As illustrated in FIG. 4, the SCU (140) may include a number of components. A power source (145) is configured to output voltage used to supply the various components within the SCU (140) with power. The power source (145) may be a primary battery, a rechargeable battery, a capacitor, or any other suitable power source. A coil (148) is configured to receive and/or emit a magnetic field (also referred to as a radio frequency (RF) field) that is used to communicate with or receive power from one or more external devices (151, 153, 155). Such communication and/or power transfer may include, but is not limited to, transcutaneously receiving data from the external device, transmitting data to the external device, and/or receiving power used to recharge the power source (145).

For example, an external battery charging system (EBCS) (151) may provide power used to recharge the power source (145) via an RF link (152). External devices including, but not limited to, a hand held programmer (HHP) (155), clinician programming system (CPS) (157), and/or a manufacturing and diagnostic system (MDS) (153) may be configured to activate, deactivate, program, and test the SCU (140) via one or more RF links (154, 156). One or more of these external devices (153, 155, 157) may also be used to control the infusion of one or more drugs into the damaged nerve to promote nerve regeneration. The CPS (157) may communicate with the HHP (155) via an infrared (IR) link (158) or via any other suitable communication link. Likewise, the MDS (153) may communicate with the HHP (155) via an IR link (159) or via any other suitable communication link.

The HHP (155), MDS (153), CPS (157), and EBCS (151) are merely illustrative of the many different external devices that may be used in connection with the SCU (140). Furthermore, it will be recognized that the functions performed by the HHP (155), MDS (153), CPS (157), and EBCS (151) may be performed by a single external device. One or more of the external devices (153, 155, 157) may be embedded in a seat cushion, mattress cover, pillow, garment, belt, strap, pouch, or the like.

The SCU (140) may also include electrical circuitry (144) configured to produce electrical stimulation pulses that are delivered to the nerve via the electrodes (142). In some embodiments, the SCU (140) may be configured to produce monopolar stimulation. The SCU (140) may alternatively or additionally be configured to produce bipolar stimulation. The electrical circuitry (144) may include one or more processors configured to decode stimulation parameters and generate the stimulation pulses. The electrical circuitry (144) may include additional circuitry such as capacitors, integrated circuits, resistors, coils, and the like configured to perform a variety of functions as best serves a particular application.

The SCU (140) may also include a programmable memory unit (146) for storing one or more sets of data and/or control parameters. The control parameters may include, but are not limited to, electrical stimulation parameters and drug stimulation parameters. The programmable memory (146) allows a patient, clinician, or other user of the SCU (140) to adjust the control parameters such that the electrical stimulation and/or drug stimulation are at levels that are safe and efficacious for a particular nerve injury and/or for a particular patient. Electrical stimulation and drug stimulation parameters may be controlled independently. However, in some instances, the electrical stimulation and drug stimulation parameters are coupled, e.g., electrical stimulation may be programmed to occur only during drug stimulation. The programmable memory (146) may be any type of memory unit such as, but not limited to, random access memory (RAM), static RAM (SRAM), a hard drive, or the like.

The electrical stimulation parameters may control various parameters of the stimulation current applied to a nerve including, but not limited to, the frequency, pulse width, and amplitude of the stimulation current. The drug stimulation parameters may control various parameters including, but not limited to, the amount of drugs infused into the nerve, the rate of drug infusion, and the frequency of drug infusion.

Different electrical stimulation and drug stimulation parameters may have different effects on nerve regeneration. Thus, in some embodiments, the electrical stimulation and/or drug stimulation parameters may be adjusted by the patient, a clinician, or other user of the SCU (140). The electrical stimulation and/or drug stimulation parameters may also be automatically adjusted by the SCU (140), as will be described below. For example, the amplitude of the stimulus current applied to a nerve may be adjusted to have a relatively low value to target relatively large diameter fibers of a peripheral nerve. The SCU (140) may also increase excitement of a nerve by applying a stimulation current having a relatively low frequency to the nerve (e.g., less than 100 Hz). The SCU (140) may also decrease excitement of a nerve by applying a relatively high frequency to the nerve (e.g., greater than 100 Hz). The SCU (140) may also be programmed to apply the stimulation current to a nerve intermittently or continuously.

As shown in FIG. 4, a pump (147) may also be included within the SCU (140). The pump (147) is configured to store and dispense one or more drugs through a catheter (143). The catheter (143) is coupled at a proximal end to the SCU (140) and may have a discharge portion (149) for infusing dosages of the one or more drugs into a predetermined site within a nerve. In some embodiments, the SCU (140) may include multiple catheters (143) and/or pumps (147) for storing and infusing dosages of the one or more drugs into predetermined sites within the nerve.

The pump or controlled drug release device described herein may include any of a variety of different drug delivery systems. Controlled drug release devices based upon a mechanical or electromechanical infusion pump may be used. In other examples, the controlled drug release device can include a diffusion-based delivery system, e.g., erosion-based delivery systems (e.g., polymer-impregnated with drug placed within a drug-impermeable reservoir in communication with the drug delivery conduit of a catheter), electrodiffusion systems, and the like. Another example is a convective drug delivery system, e.g., systems based upon electroosmosis, vapor pressure pumps, electrolytic pumps, effervescent pumps, piezoelectric pumps and osmotic pumps.

Exemplary controlled drug release devices suitable for use as described herein include, but are not necessarily limited to, those disclosed in U.S. Pat. Nos. 3,760,984; 3,845,770; 3,916,899; 3,923,426; 3,987,790; 3,995,631; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 4,911,616; 5,057,318; 5,059,423; 5,085,562; 5,112,614; 5,137,727; 5,219,278; 5,224,843; 5,234,692; 5,234,693; 5,271,724; 5,277,556; 5,728,396; 5,759,014; 5,759,015; 6,368,315; 6,464,687; 2004/0082908 and the like. All of these listed patents are incorporated herein by reference in their respective entireties.

The SCU (140) of FIG. 4 may be implanted within the patient (150) using any suitable surgical procedure such as, but not limited to, injection, small incision, open placement, laparoscopy, or endoscopy. In some instances, the SCU (140) may be implanted at a site that is any distance from a treatment site with the lead (141) and/or the catheter (143) being routed to the treatment site. The treatment or target site is the site to which electrical stimulation and/or drug stimulation is to be applied to promote nerve regeneration. The SCU (140) may also or alternatively be implanted at or near the treatment site, as will be described in more detail below.

The SCU (140) of FIG. 4 is illustrative of the many types of SCUs that may be used to apply electrical stimulation to a nerve and/or infuse one or more drugs into the nerve to promote nerve regeneration. For example, the SCU (140) may include an implantable pulse generator (IPG) coupled to one or more leads (141) having a number of electrodes (142). In the case of drug stimulation only, the SCU (140) comprises a pump. Alternatively, the SCU (140) may be an implantable microstimulator, such as a BION® microstimulator (Advanced Bionics® Corporation, Valencia, Calif.). The following listed patents describe various details associated with the manufacture, operation, and use of BION implantable microstimulators, and are all incorporated herein by reference:

| Application/Patent/ | Filing/ | |
|---|---|---|
| Publication No. | Publication Date | Title |
| U.S. Pat. No. | Issued | Implantable Microstimulator |
| 5,193,539 | Mar. 16, 1993 | |
| U.S. Pat. No. | Issued | Structure and Method of |
| 5,193,540 | Mar. 16, 1993 | Manufacture of an Implantable |
| | | Microstimulator |
| U.S. Pat. No. | Issued | Implantable Device Having an |
| 5,312,439 | May 17, 1994 | Electrolytic Storage Electrode |
| U.S. Pat. No. | Issued | Battery-Powered Patient |
| 6,185,452 | Feb. 6, 2001 | Implantable Device |
| U.S. Pat. No. | Issued | System of Implantable Devices |
| 6,164,284 | Dec. 26, 2000 | For Monitoring and/or Affecting |
| | | Body Parameters |
| U.S. Pat. No. | Issued | System of Implantable Devices |
| 6,208,894 | Mar. 27, 2001 | For Monitoring and/or Affecting |
| | | Body Parameters |
| U.S. Pat. No. | Issued | Implantable Microstimulator |
| 6,051,017 | Apr. 18, 2000 | and Systems Employing Same |

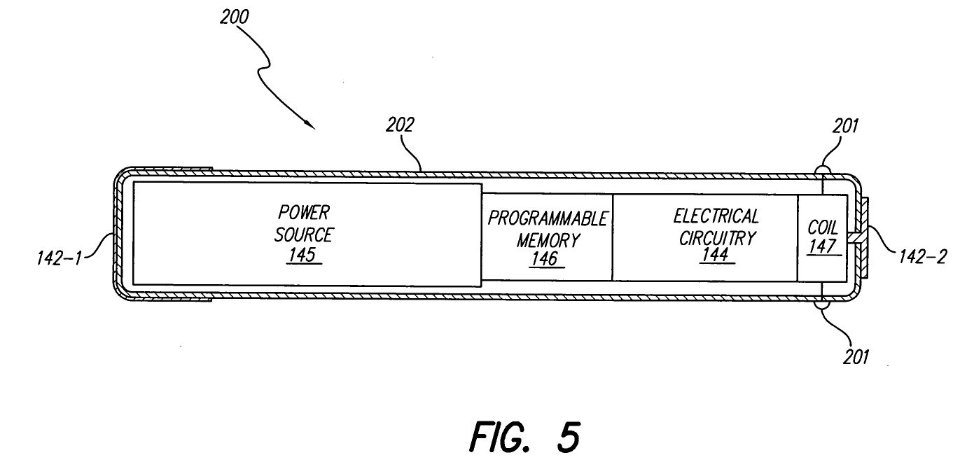

FIG. 5 illustrates an exemplary BION microstimulator (200) that may be used as the SCU (140; FIG. 4) described herein. Other configurations of the microstimulator (200) are possible, as shown in the above-referenced patents and as described further below.

As shown in FIG. 5, the microstimulator (200) may include the power source (145), the programmable memory (146), the electrical circuitry (144), and the pump (147) described in connection with FIG. 4. These components are housed within a capsule (202). The capsule (202) may be a thin, elongated cylinder or any other shape as best serves a particular application. The shape of the capsule (202) may be determined by the structure of the desired target, the surrounding area, and the method of implementation. For example, the diameter of the capsule (202) may be less than 5 millimeters (mm) and the length of the capsule (202) may be less than 40 mm in some instances. It will be recognized that the diameter, width, and/or length of the capsule (202) may be any size.

The microstimulator (200) may be implanted within a patient with a surgical tool such as a hypodermic needle or any other tool specially designed for the purpose. Alternatively, the microstimulator (200) may be implanted using endoscopic or laparoscopic techniques.

As shown in FIG. 5, the microstimulator (200) may include one or more infusion outlets (201) to which one or more catheters (not shown) may be attached. The infusion outlets (201) facilitate the infusion of one or more drugs into a treatment site to immediately and/or ultimately promote nerve regeneration. The stimulator (200) of FIG. 5 also includes electrodes (142-1 and 142-2) at either end of the capsule (202). One of the electrodes (142) may be designated as a stimulating electrode to be placed close to the treatment site and one of the electrodes (142) may be designated as an indifferent electrode used to complete a stimulation circuit.

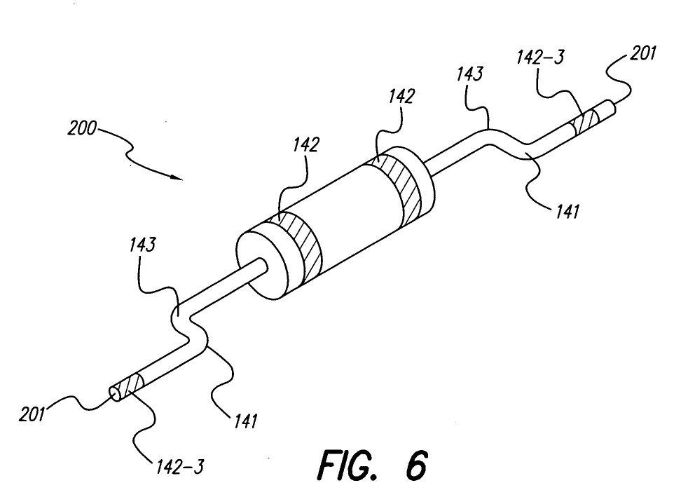

FIG. 6 shows that one or more catheters (143) may be coupled to the microstimulator (200). Infusion outlets (201) may be located at the end of a catheter (143) to facilitate drug infusion. As shown in FIG. 6, the catheters (143) may also serve as leads (141) having one or more electrodes (142-3). Thus, the catheters (143) and leads (141) of FIG. 6 permit infused drugs and/or electrical stimulation to be directed to a treatment site while allowing most elements of the microstimulator (200) to be located in a surgically convenient site.

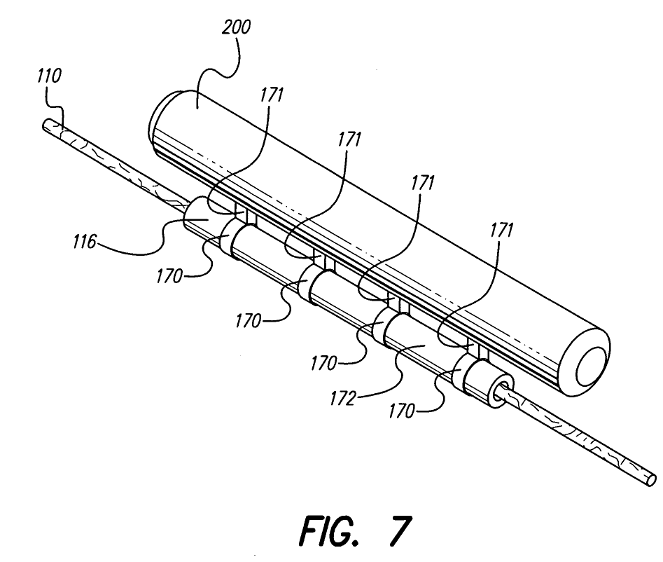

Referring again to FIG. 5, the microstimulator (200) may be attached or implanted adjacent to a nerve or other treatment site. For example, FIG. 7 shows a BION microstimulator (200) that is coupled to a nerve graft (116). The microstimulator (200) is coupled to a nerve graft (116) for illustrative purposes only. It will be recognized that the microstimulator (200) may be coupled directly to the nerve (200), as opposed to a nerve graft, in a similar manner.

As shown in FIG. 7, the microstimulator (200) is positioned parallel with the nerve graft (116). The nerve graft (116) may be, for example, a collagen tube impregnated with Schwann cells, stem cells, and/or any other growth factors that promote nerve regeneration. The microstimulator (200) may include one or more connectors (171) configured to electronically couple the electrical circuitry (144; FIG. 5) to one or more moveable electrodes (170). The connectors (171) may also or alternatively be configured to couple the microstimulator (200) to the nerve graft (116) or to the nerve itself. In some embodiments, the moveable electrodes (170) may be selectively moved to any location along the nerve graft (116). For example, the electrodes (170) may be initially positioned at or near the ends of the nerve graft (116) and then gradually moved towards the center of the nerve graft (116), thereby promoting the growth and eventual rejoining of the severed nerve (110).

Numerous methods may be employed to move the electrodes (170). For example, the electrodes (170) may be periodically repositioned via a surgical procedure. Alternatively, a spring mechanism (not shown) or the like may be coupled to the electrodes (170), the connectors (171), and/or the microstimulator (200) such that the natural resting position of the electrodes (170) is near the center of the nerve graft (116). A biodegradable substance may be applied to the outer edge of the nerve graft (116) or to the electrodes (170) after the electrodes (170) have been positioned at or near the end of the nerve graft (116). The biodegradable substance may be designed to impede the electrodes (170) from returning to their natural resting positions. With time, the biodegradable substance gradually decomposes and disappears, thereby allowing the electrodes (170) to gradually return to their natural resting positions.

As shown in FIG. 7, an axial wire (172) may be coupled to the nerve graft (116) along a desired nerve growth path. The axial wire (172) may be coated to create passive electrodes used by the microstimulator (200) to further promote nerve regeneration along the desired nerve growth path. The system and method of coupling the microstimulator (200) to the nerve graft (116) and/or nerve (110) illustrated in FIG. 7 is merely exemplary of the many different methods and systems that may be used to couple the microstimulator (200) to the nerve graft (116) and/or nerve (110).

The SCU (140) may be configured to operate independently. Alternatively, the SCU (140) may be configured to operate in a coordinated manner with one or more additional SCUs (140), other implanted devices, or other devices external to the patient's body. For instance, a first SCU (140) may control or operate under the control of a second SCU (140), other implanted device, or other device external to the patient's body. The SCU (140) may be configured to communicate with other implanted SCUs (140), other implanted devices, or other devices external to the patient's body via an RF link, an untrasonic link, an optical link, or any other type of communication link. For example, the SCU (140) may be configured to communicate with an external remote control that is capable of sending commands and/or data to the SCU (140) and that is configured to receive commands and/or data from the SCU (140).

In order to determine the amount and/or type(s) of stimulating drug(s) and/or the strength and/or duration of electrical stimulation required to most effectively promote nerve regeneration, a patient's response to and/or need for treatment may be sensed. For example, the amount of nerve regeneration, activity in the target nerve, or symptoms thereof (e.g., neurotransmitter levels, target organ stimulation, etc.) may be sensed or measured. Other characteristics of the patient including, but not limited to, hormone levels and electromyography (EMG) signal levels may also be sensed or measured. In some embodiments, the SCU (140) may be configured to change the stimulation and/or drug stimulation parameters in a closed loop manner in response to these measurements. The SCU (140) may be configured to perform the measurements. Alternatively, other measuring devices may be configured to perform the measurements and transmit the measured values to the SCU (140).

For example, the SCU (140) may be implanted adjacent to the pudendal and/or cavernous nerves to promote nerve regeneration after prostate surgery. The SCU (140) may include one or more sensing devices configured to sense changes in the patient in response to electrical stimulation and/or drug stimulation. Other measures of the state of the patient may additionally or alternatively be sensed by the sensing devices, e.g., cavernous nerve firing rate; intercavernous pressure; joint angle; muscle activity (e.g., EMG); nerve activity (e.g., ENG); and/or other measures. The sensing device may be a pressure sensor such as a penile tumescence sensor or penile arteriole pressure sensor, for example. The SCU (140) may be configured to change the stimulation and/or drug stimulation parameters in response to any of the above mentioned measurements in a closed loop manner.

As mentioned, the sensing device may be included in the SCU (140). Alternatively, the sensing device may be a separate device that is implanted in or near a nerve or other organ. For example, a sensing device may be implanted in or around the penis or its internal structures.

The SCU (140) may be further configured to provide electrical stimulation and/or drug stimulation of a nerve after the nerve has completely regenerated. For example, the SCU (140) may be configured to provide electrical stimulation and/or drug infusion to the cavernous nerve and/or the pudendal nerve in order to effect erection of the penis.

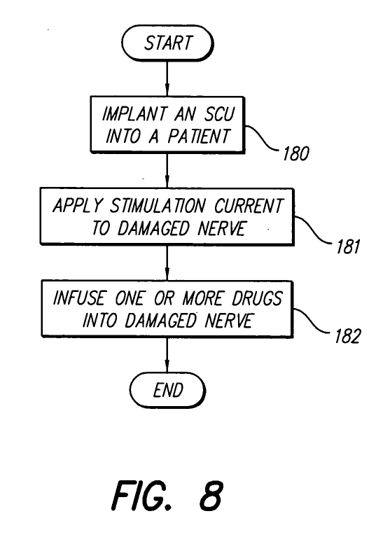

FIG. 8 is a flow chart illustrating an exemplary method of regenerating a damaged nerve within a patient. The method of FIG. 8 is merely exemplary of the many different methods that may be used to promote regeneration of a damaged nerve and may be modified as best serves a particular application. As shown in FIG. 8, an SCU (140) is first implanted within a patient (step 180). The SCU (140) may be implanted using any suitable surgical procedure such as, but not limited to, injection, small incision, open placement, laparoscopy, or endoscopy. The SCU (140) may be coupled to the nerve and/or a nerve graft (116; FIG. 7) using the method described in connection with FIG. 7 or using any other suitable method. Alternatively, the SCU (140) may be implanted in a location distant from the treatment site. In these instances, the lead(s) (141) and/or the catheter(s) (143) may be routed to the treatment site.

Once the SCU (140) is implanted into a suitable location within the patient (step 180), stimulation current may be applied to the damaged nerve (step 181). One or more drugs may also or alternatively be infused into the damaged nerve (step 182). The stimulation current and/or the one or more drugs may be applied to any portion of the damaged nerve, an organ (113; FIG. 3) connected to the nerve, a nerve graft (116) corresponding to the nerve, or any other tissue within the patient to which applied stimulation current and/or drugs may promote regeneration of the damaged nerve. In some embodiments, the implanted SCU (140) applies the stimulation current (step 181) and infuses the drugs into the damaged nerve (step 182). In alternative embodiments, the stimulation current and/or drugs are applied to the damaged nerve by a second implanted device. The second implanted device may be a second SCU (140) or any other implanted device.

The preceding description has been presented only to illustrate and describe embodiments of invention. It is not intended to be exhaustive or to limit the invention to any precise form disclosed. Many modifications and variations are possible in light of the above teaching. It is intended that the scope of the invention be defined by the following claims.

What is claimed is:

1. A method of regenerating a nerve within a patient, said method comprising:
implanting a system control unit within said patient; and
applying a stimulus to said nerve with said system control unit in accordance with one or more control parameters;
wherein said stimulus promotes said regeneration of said nerve.

2. The method of claim 1, wherein said system control unit is coupled to one or more electrodes, and wherein said stimulus comprises a stimulation current delivered via said electrodes.

3. The method of claim 2, wherein said control parameters control one or more of a frequency of said stimulation current, a pulse width of said stimulation current, and an amplitude of said stimulation current.

4. The method of claim 1, wherein said system control unit is connected to at least one catheter, and wherein said stimulus comprises stimulation via one or more drugs delivered through said at least one catheter.

5. The method of claim 4, wherein said control parameters control one or more of an amount of said one or more drugs delivered through said at least one catheter and a rate of delivery of said one or more drugs through said at least one catheter.

6. The method of claim 5, wherein said one or more drugs comprise at least one or more of a neurotrophic factor, a nerve growth factor, a brain-derived neurotropic factor, a Schwann cell product, a neurotrophic tyrosine kinase type two (TrkB), a protein kinase A (PKA), and a L2/HNK-1 carbohydrate.

7. The method of claim 1, wherein said system control unit is coupled to one or more electrodes and to at least one catheter, and wherein said stimulus comprises a stimulation current delivered via said electrodes and stimulation via one or more drugs delivered through said at least one catheter.

8. The method of claim 1, further comprising sensing at least one condition and using said at least one sensed condition to automatically adjust one or more of said control parameters.

9. The method of claim 8, wherein said at least one sensed condition is at least one or more of a neurotransmitter level, a nerve regeneration measurement, a hormone level, an electromyography signal level, a change in penile tumescence, a change in penile arteriole pressure, and a response of said patient to said stimulus.

10. The method of claim 1, further comprising manually adjusting said control parameters.

11. The method of claim 1, wherein said step of implanting said system control unit within said patient comprises coupling said system control unit to said nerve using one or more connectors.

12. The method of claim 1, wherein said system control unit comprises a microstimulator.

13. The method of claim 1, wherein said nerve comprises a nerve graft.

14. The method of claim 1, wherein said nerve is at least one or more of a cavernous nerve, a pudendal nerve, and a brachial plexus nerve.

15. The method of claim 1, wherein said stimulus is a treatment for erectile dysfunction.

16. The method of claim 1, further comprising communicating with or transferring power to said system control unit using an external device.

17. A system for regenerating a nerve within a patient, said system comprising:
a system control unit configured to apply a stimulus to said nerve in accordance with one or more control parameters;
wherein said system control unit is implanted within said patient and said stimulus promotes said regeneration of said nerve.

18. The system of claim 17, further comprising two or more electrodes coupled to said system control unit, wherein said stimulus comprises a stimulation current delivered by said system control unit via said electrodes.

19. The system of claim 18, wherein said control parameters control one or more of a frequency of said stimulation current, a pulse width of said stimulation current, and an amplitude of said stimulation current.

20. The system of claim 17, further comprising a pump for delivering one or more drugs, said pump coupled to a catheter, and wherein said stimulus comprises stimulation via said one or more drugs delivered through said catheter.

21. The system of claim 20, wherein said control parameters control one or more of an amount of said one or more drugs delivered through said catheter and a rate of delivery of said one or more drugs through said catheter.

22. The system of claim 20, wherein said one or more drugs comprise at least one of a neurotrophic factor, a nerve growth factor, a brain-derived neurotropic factor, a Schwann cell product, a neurotrophic tyrosine kinase type two (TrkB), a protein kinase A (PKA), and a L2/HNK-1 carbohydrate.

23. The system of claim 17, further comprising:
two or more electrodes coupled to said system control unit; and
a pump for delivering one or more drugs, said pump coupled to a catheter;
wherein said stimulus comprises a stimulation current delivered by said system control unit via said electrodes and stimulation via said one or more drugs delivered by said pump.

24. The system of claim 17, further comprising:
a sensor device for sensing at least one condition;
wherein said system control unit uses said at least one sensed condition to automatically adjust one or more of said control parameters.

25. The system of claim 24, wherein said at least one sensed condition is at least one or more of a neurotransmitter level, a nerve regeneration measurement, a hormone level, an electromyography signal level, a change in penile tumescence, a change in penile arteriole pressure, and a response of said patient to said stimulus.

26. The system of claim 17, wherein said control parameters are manually adjusted.

27. The system of claim 17, wherein said system control unit further comprises a programmable memory unit configured to store said control parameters.

28. The system of claim 17, wherein said system control unit comprises a micro stimulator.

29. The system of claim 28, wherein said microstimulator is coupled to said nerve with one or more connectors.

30. The system of claim 29, further comprising two or more moveable electrodes coupled to said microstimulator, wherein said stimulus comprises a stimulation current delivered to said nerve via said moveable electrodes.

31. The system of claim 30, wherein said nerve includes a nerve graft, and wherein said moveable electrodes are coupled to said nerve graft.

32. The system of claim 31, wherein said moveable electrodes are configured to move from an outer portion of said nerve graft towards a center portion of said nerve graft.

33. The system of claim 17, wherein said nerve comprises a nerve graft.

34. The system of claim 17, wherein said nerve is at least one or more of a cavernous nerve, a pudendal nerve, and a brachial plexus nerve.

35. The system of claim 17, wherein said system control unit is further configured to promote an erection of a penis.

36. The system of claim 17, further comprising an external device configured to communicate with or transfer power to said system control unit.

37. A system for regenerating a nerve within a patient, said system comprising:
means for generating a chemical or electrical stimulus; and
means for applying said stimulus to said nerve with said system control unit in accordance with one or more control parameters;
wherein said stimulus promotes said regeneration of said nerve.

38. The system of claim 37, wherein said system control unit is coupled to one or more electrodes, and wherein said stimulus comprises a stimulation current delivered via said electrodes.

39. The system of claim 37, wherein said system control unit is coupled to a means for delivering one or more drugs to said nerve, and wherein said stimulus comprises stimulation via delivery of said one or more drugs to said nerve.

40. The system of claim 37, further comprising means for sensing at least one condition and means for using said at least one sensed condition to automatically adjust one or more of said control parameters.

41. The system of claim 37, further comprising means for manually adjusting said control parameters.

42. The system of claim 37, wherein said means for implanting said system control unit within said patient comprises means for coupling said system control unit to said nerve.

43. The system of claim 37, further comprising means for communicating with or transferring power to said system control unit.
